(19) United States
(12) Patent Application Publication
(10) Pub. No.: US 2008/0097202 A1
(43) Pub. Date: Apr. 24, 2008

(54) Method for Performing Elastography

(76) Inventors: Jean-Yves Chapelon, Villeurbanne (FR); Remi Souchon, Vienne (FR)

Correspondence Address: MARSH, FISCHMANN & BREYFOGLE LLP, 3151 SOUTH VAUGHN WAY, SUITE 411, AURORA, CO, 80014, US

(21) Appl. No.: 11/571,650

(22) PCT Filed: Jul. 5, 2005
(86) PCT No.: PCT/EP05/07912

§ 371 Date: Jan. 4, 2007

(30) Foreign Application Priority Data
Jul. 9, 2004 (EP) 04291758.3

(51) Int. Cl. A61B 8/00

(52) U.S. Cl. 600/437

(57) ABSTRACT

A method for performing elastography is provided, comprising the following steps. First, emitting a first coded signal towards tissue and recording an echoed signal in response. Next, applying a strain on the tissue, such as a compression or an expansion. Then, emitting a second coded signal towards the tissue under strain, said second signal being a compressed (or stretched) version of the first emitted signal in the time domain, and recording an echoed signal in response. Then processing the second echoed signal by stretching (or compressing) it in the time domain by a factor matched to the compression (or stretching) factor that was applied to the second emitted signal. Finally, cross-correlating the first echoed signal and the processed second echoed signal to provide an elastographic image of said tissue. The method of the invention uses different emitted signals before and during strain applied to the tissue. The image quality in elastography can therefore be considerably improved.

The invention relates to a method for performing elastographic imaging of a target body.

Ultrasonic elastography is a system for measuring and imaging strain distributions in an elastic tissue. This system is typically based on external compression of a target body, and utilizes one or more transducers, acting as or with a compressor, to generate pre- and post-compression sonic pulses and receive the resulting echo sequences from within the target body. The pre- and post-compression echo sequence pairs may then be cross-correlated or matched to determine the strain along the path of the sonic pulses, and preferably to yield a strain profile of the target body. This strain profile may then be converted into a compressibility profile by measuring the stress imposed by the compressing device and calculating the elastic moduli based on the stress and the strain profile.

Elastography has become a medical imaging technique involving evaluating internal deformation undergone by tissue during compression in order to determine elasticity of said tissue and therefore eventually detect abnormal masses. This technique can be used for cancer screening for instance.

Elastography method and apparatus are described in U.S. Pat. No. 5,474,070.

What we refer to by the term elastographic image or elastogram is the strain distribution on a spatial basis. The spatial basis may be defined as lateral scanning versus depth. The depth can be given by time travel of the signals, i.e. time range between the pulse emission and the echoed reception. Lateral scanning represents the surface portion of tissue to be analyzed. The image therefore gives a two-dimensional representation of a given surface of tissue.

Image quality in elastography can be estimated by quantities such as elastographic signal-to-noise ratio (SNRe) and axial resolution (Ra). SNRe is defined as the mean to standard deviation ratio of the measured strain in an area of uniform strain. Ra is defined as the smallest distance between two resolvable targets.

It has been shown that high cross-correlation values are required between the pre-compression signal and the post-compression signal in order to obtain a precise estimation of the displacement, hence precise strain estimates.

In elastography, correlation is degraded by two factors: increasing strain and decreasing sonographic signal-to-noise ratio (SNRs). Because correlation degrades with increasing strain, there exists an upper bound that prevents elastographic acquisition for strains greater than about 1-2%. Stretching of the post-compression backscattered signal before time delay estimation has been proposed but the method only partially overcomes this degradation. Regarding the post-compression signal stretching, it can be referred to the publications “Noise reduction in elastograms using temporal stretching with multicompression averaging”, by T. Varghese, J. Ophir and I. Cespedes, Ultrasound in Med & Biol, Vol. 22, No. 8, pp 1043-1052, 1996; “An adaptative strain estimator for elastography”, by S K. Alam, J. Ophir, E. Konofagou, IEEE Trans Ultrason Ferroelectr Freq Control, Vol. 45, No. 2, pp 461-472, 1998 and to patents U.S. Pat. No. 6,514,204 and U.S. Pat. No. 6,277,074.

In the article “Deformation models and correlation analysis in elastography” by Bilgen M and Insana M F, J. Acoust. Soc. Am. 99 (5), 3212-3224 (1996), Bilgen and Insana showed how to shape the post-compression point spread function (PSF) in addition to stretching the post-compression echo signal, in the case of a Gaussian pulse, in order to increase correlation. The PSF of an ultrasound system is the image of a single point scatterer. The PSF depends on the electromechanical properties of the transducer(s), on the excitation voltage applied to the transducers(s). It may be affected by the acoustic properties of the medium being investigated, for example by frequency-dependant attenuation. The dimension of the PSF along the axis of propagation of the ultrasound signal is given by the duration of the echoed signal received from the target point, and is often measured as the full width at half maximum (FHWM) of the said echoed signal.

SNRs is defined as the ratio of the ultrasonic signal power over the noise power at a given spatial location. High SNRs is required to achieve high correlation. It can be obtained by increasing the amplitude or the duration of the emitted signal. In biological tissues the maximal signal amplitude is limited by the Mechanical Index (MI) that can be applied before cavitation-induced damage starts to occur. Most ultrasound scanners already operate at this level, so that pulse amplitude cannot be further increased. Increasing pulse duration using bursts provides increased SNRs but degrades the axial resolution of the ultrasonic system.

This problem of image quality was previously identified in sonographic imaging. Usual sonography systems have chosen to use short pulse waves to favor axial resolution, and perform signal processing to deal with SNRs.

Alternatively, emitting long coded excitation signal was shown to increase SNRs in conventional sonography and in Doppler imaging without impairing spatial resolution. Regarding long coded ultrasonic excitation signals, it can be referred to the publication “Coded excitation for diagnostic ultrasound: A system developer's perspective”, by R Y. Chiao and X. Hao, Proceeding IEEE Ultrasonics Symposium 2003, Vol. 1, pp 437-448 and to U.S. Pat. No. 6,213,947.

It is an object of the invention to propose an elastography method that may totally overcome the image degradation due to strain, and that minimizes image degradation due to SNRs without impairing axial resolution.

According to the invention a new elastography method is provided using different point spread functions (PSF) and coded excitation before and during compression of the tissues.

In particular, the invention concerns a method for performing elastography, the method comprising the steps of:

a) applying of a first coded voltage waveform u₁(t) to the transducer(s) to emit a first ultrasonic signal p₁(t) towards tissue, with p₁(t)=u₁(t)*h(t) being the PSF generated by the waveforms u₁, where t denotes time and symbol * denotes a convolution product, h(t) being the impulse response of the transducer;

b) recording a first echoed signal s₁(t) in response to said first emitted signal;

c) applying a strain on the tissue;

d) applying of a second coded voltage waveform u₂(t) to the transducer(s) to emit a second ultrasonic signal p₂(t) towards the tissue under strain, with p₂(t)=u₂(t)*h(t) being the PSF generated by the waveforms u₂, said second emitted signal being linked to said first emitted signal with the following relation:

p₂(t)=p₁(αt),

with α=1/(1+68 ), ε being an initial estimate of the strain applied to the tissue;

e) recording a second echoed signal s₂(t) in response to said second emitted signal;

f) processing said second echoed signal according to the following relation:

s₂″(t)=s₂(t/α);

g) cross-correlating the first echoed signal s₁(t) and the processed second echoed signal s″₂(t) to provide an elastographic image of said tissue.

According to embodiments of the invention, the method comprises one or several of the following features:

the strain applied to the tissue in step c) is negative, providing a compression of said tissue;

the strain applied to the tissue in step c) is positive, providing an expansion of said tissue;

the first and second emitted signals are burst signals;

the first emitted signal is pre-stretched so that its point spread function duration is increased.

According to one embodiment, the second signal p₂(t) is emitted with a predetermined value of α in step d), providing an elastographic image in step g), the method further comprising the steps of:

h) calculating a local strain ε applied on tissue from the results of said elastographic image;

i) adjusting the value of a according to the calculated local strain ε;

j) repeating steps d) to g) with an adjusted value of α.

According to one embodiment, steps h) to j) are repeated iteratively until the adjusted value of α is substantially equal to the value applied to the second emitted signal p₂(t).

The invention also concerns a system for performing the elastography method according to the invention, comprising:

hardware or computer-implemented software to link the second signal to be emitted to the first emitted signal;

hardware or computer-implemented software to cross-correlate the echoed signals.

According to embodiments, the system may further comprise:

hardware or computer-implemented software to calculate a local strain ε applied on tissue and adjust the value of a accordingly; and/or

hardware or computer-implemented software to code the emitted signals.

Other characteristics and advantages of the invention will appear on reading the following detailed description of some embodiments of the invention given solely as an example and taken in connection with the accompanying drawings in which:

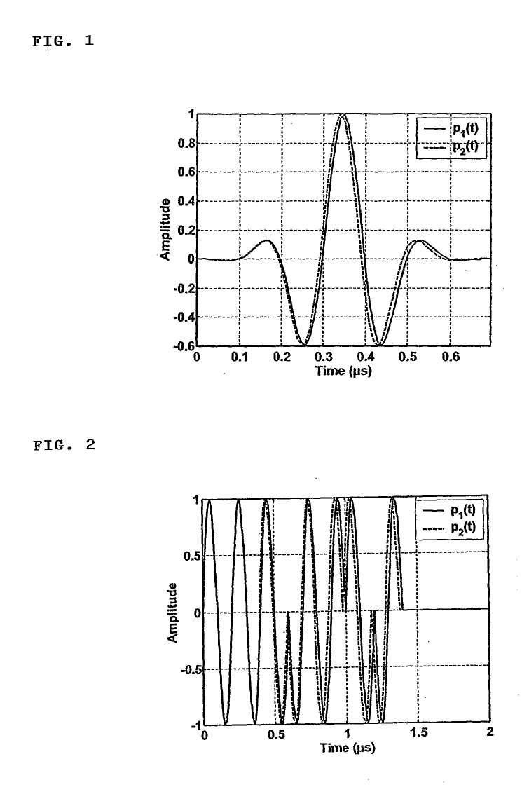

FIG. 1 is a graph showing pre- and post-compression short pulse emitted signals;

FIG. 2 is a graph showing pre- and post-compression Barker coded emitted signals.

The invention concerns a method where the pre- and post-compression emitted signals have different characteristics, these characteristics being chosen depending on the strain to be measured.

According to the invention, a new elastography method is provided comprising the following steps. First, emitting a first coded signal towards tissue and recording a first echoed signal in response to said first signal. Next, applying a strain on the tissue, such as a compression or an expansion, preferably of a known value. The strain applied to the tissue could also alternate compression and expansion. Then, emitting a second coded signal towards the tissue under strain, said second signal being a compressed (or stretched) version of the first emitted signal in the time domain, and recording a second echoed signal in response to said second signal. Then processing the second echoed signal by stretching (or compressing) it in the time domain by a factor matched to the compression or stretching factor that was applied to the second emitted signal. Finally, cross-correlating the first echoed signal and the processed second echoed signal to provide an elastographic image of said tissue.

The time-domain stretching or compression of the second emitted signal depends on the strain applied to the tissue, i.e. if the tissue is compressed (respectively stretched by expansion), the second emitted signal will be a compressed version (respectively a stretched version) of the first emitted signal.

According to the invention, the elastography method uses different emitted signals before and after strain is applied to the tissue. In particular, the invention concerns a method for which the shape of the post-compression signal is a compressed (or stretched) version of the pre-strain signal. The compression (or stretching) factor a can be matched to an estimate of the applied strain ε. The pre- and post-compression signals are coded signals; it can be bursts coded signals.

The use of coded excitation allows further improvement in image quality in areas where the sonographic signal-to-noise ratio (SNRs) is low, without any loss in axial resolution.

According to the invention, an ultrasonic signal is emitted towards tissue, being firstly at rest. As well known from sonography, an echoed signal is returned from the tissue. Such echoed signal can be recorded and analyzed to provide information on the tissue condition.

The ultrasonic pulse p(t) emitted towards tissue can be modeled as follows:

p(t)=u(t)*h(t)

where t denotes time, and

with, u(t) being the electric pulse applied to the ultrasonic transducer
h(t) being the transducer impulse response
the symbol * being the convolution product

The echoed signal s(t) can be modeled as follows:

s(t)=p(t)*m(t)+η(t)

with, m(t) being the reflectivity function of the scatterer distribution that model the tissue, and η(t) being additive electronic noise.

If noise is neglected, the echoed signal recorded before strain is applied to the tissue is

s₁(t)=p₁(t)*m(t);

and the echoed signal recorded while applying a strain to the tissue is

s₂(t)=p₂(t)*m(αt);

with α=1/(1+ε) linked to the uniform strain s applied to the tissue. If ε is positive, the tissue is expanded and if ε is negative, the tissue is compressed. p₁(t) and p₂(t) are respectively the pre- and post-compression emitted ultrasonic pulses.

The cross-correlation in the frequency domain of said echoed signals before and during strain is formulated as such:

C(f)=S₁(f) S₂*(f)

with S₁(f) and S₂(f) being the Fourier Transform of the respective echoed signals s₁(t) and s₂(t), and * being the complex conjugation.

In conventional sonography, the emitted pre and post-compression pulses p₁(t) and p₂(t) are identical. The cross-correlation function can be expressed as follows:

C(f)=(1/|α|) ∥P₁(f)∥² M(f)M*(f/α)

with M(f) being the Fourier Transform of the reflectivity function m(t). Correlation degrades with increasing strain because the M(f)M*(f/α) product is lower that the autocorrelation function ∥M(f)∥² of the tissue.

Uniform or local stretching was proposed to recover the autocorrelation function of the tissue. It can be referred to the previously cited publications of T. Varghese, 1996; S K. Alam, 1998 and patents U.S. Pat. No. 6,514,204 and U.S. Pat. No. 6,277,074.

The principle of these methods is to stretch the post-compression echoed signal s₂(t) by a factor a in the time domain, i.e. to define s₂′(t) as follows:

s₂′(t)=s₂(t/α)=p₂(t/α)*m(t)/α

Then the cross-correlation between the pre-compression signal s₁(t) and the stretched post-compression signal s₂′(t) is, in the frequency domain:

C(f)=(1/|α|) P₁(f) P₁*(αf) ∥M(f)∥²

This method recovers the autocorrelation function of the tissues but at the cost of degrading the autocorrelation function of the transmitted pulse ∥P₁(f)∥². This is because the point-spread function (PSF) is also stretched with this method.

However, according to the invention, the pre- and post-strain signals are not identical, i.e. the duration of the second emitted pulse is stretched or compressed compared to the duration of the first emitted pulse.

The post-compression pulse p₂(t) is compressed in the time domain compared to the pre-compression pulse p₁(t) and can be expressed as follows:

p₂(t)=p₁(αt).

This is achieved using different excitation voltage waveforms u₁(t) and u₂(t) that are related by: u₂(t)*h(t)=α u₁(αt)*h(αt).

Knowledge of the impulse response h(t) is necessary to apply this equation. In practice, a difficulty arises because the spectrum H(f) of the impulse response h(t) can be measured accurately only within a limited frequency range. A solution is to fit a theoretical model, for example a Gaussian distribution, to the experimental impulse response. Alternatively, a combination of experimental data (inside the frequency range in which the impulse response can be measured accurately) and of theoretical data (outside of the range) can also be used.

Moreover, because the shortest PSF will always be achieved with a spike voltage, it is not possible to achieve compression of the post-compression PSF p₂(t) if the pre-compression PSF p₁(t) was obtained with spike excitation. However this is still possible if care was taken to pre-stretch the pre-compression PSF p₁(t) so that the post-compression PSF p₂(t)=p₁(αt) has duration greater or equal to the duration of the impulse response h(t).

FIG. 1 shows an example of emitted signals p₁(t) and p₂(t) with a factor α=1.02, corresponding to an applied compression strain ε of 2% on the tissue of the target body. The method according to the invention makes it possible to improve greatly the signal to noise ratio through an improvement in the correlation between the pre- and post-compression echoed signals.

In the example of FIG. 1, an ultrasonic transducer was used to provide pulsed excitations with a central frequency of 5 MHz. It can be seen that the signal p₂(t) is a compressed version in the time domain of p₁(t).

The method creates a time-domain compression (or expansion) of the PSF (Point Spread Function) of the imaging system, which is then restored by numerical stretching (or compression) of the echoed signal, as shown below.

The post-compression signal becomes: s₂(t)=α p₁(αt)*M(αt);

Then global or adaptive stretching of the post-compression signal is used to obtain a stretched version s₂″ of the post-compression echoed signal:

s₂″(t)=s₂(t/α)=p₁(t)*m(t)=s₁(t)

Then the cross-correlation between the pre-compression signal s₁(t) and the stretched post-compression signal s₂″(t) can be expressed as:

C(f)=S₁(f)S₂″*(f)=∥S₁(f)∥²=∥P₁(f)∥² ∥M(f)∥²

It can be noted that the cross-correlation function is equal to auto-correlation function of the pre-compression pulse. Therefore, the decorrelation noise due to the strain applied to the tissue is totally eliminated. The image quality in elastography can therefore be considerably improved by such method according to the invention.

The calculation applies regardless of the shape of the emitted signals, which can be either short pulses, burst signals, or coded waveforms.

The strain applied to the tissue may be a compression or an expansion, without impacting on the method according to the invention. The terms pre-compression, post-compression, global stretching and others, can be changed into pre-stretching, post-stretching and global compressing without changing the previous equations, as the value of the factor α depends on the type of strain applied to the tissue.

According to an embodiment of the invention, the image quality can be improved by adjusting the value α to a value as close as possible to the real strain ε applied. First, the method according to the invention is conducted by emitting a second signal p₂(t) with a predetermined value of α, as illustrated on FIG. 1. A first elastographic image is provided and the local strain ε applied on tissue can be calculated from said first elastographic image. The value of α can therefore be adjusted to the calculated strain E and further second signals p₂(t) can be emitted with an adjusted values of α to generate a new elastogram. The emitting of a post-compression signal p₂(t) can be repeated iteratively until the calculated adjusted value of α is substantially equal to the value applied to the second emitted signal p₂(t).

According to the invention, the use of coded excitation allows further improvement in image quality in areas where the sonographic signal-to-noise ratio (SNRs) is low, without any loss in axial resolution.

In this case, the pre-compression emitted signal p₁(t) can be any of the coded signals that can be used for sonography, such as a frequency-modulated chirp, a Barker code, a Golay complementary series or any pseudo-random sequences whose auto-correlation function is a Dirac function. The post-compression emitted signal p₂(t) is derived from the coded pre-compression emitted signal p₁(t) using equation p₂(t)=p₁(αt).

FIG. 2 shows an example of pre-compression emitted coded signal p₁(t) and matched post-compression emitted signal p₂(t) designed for an estimated compression strain of 2% (α=1.02) and an ultrasonic transducer with a central frequency of 5 MHz, using a Barker code of length 7. The signal p₂(t) is a compressed version in the time domain of p₁(t).

Combining coded excitation with the invention has great potential because two of the major causes of noise in displacement estimates in elastography, namely strain-induced decorrelation and SNRs-induced decorrelation, are significantly decreased.

Moreover, using coded excitation in elastography can be very simple as no matched filtering is mandatory on echoed signals before the cross-correlation of the echoed signals is estimated. Cross-correlation of the first echoed signal s₁(t) and the processed second echoed signal s″₂(t) can be conducted on coded signal without impacting the results of the elastographic image

The invention also relates to a computer system able to implement the method according to the invention. The computer system thereof comprises computer-implemented software to link the second signal to be emitted to the first emitted signal, and computer-implemented software to cross-correlate the echoed signals.

The computer system of the invention is adapted to handle the signal processing in order to define the parameters of the post-compression signal p₂(t) to be emitted responding to the criteria of the first or second embodiment of the method of the invention. In particular, the computer comprises hardware or computer-implemented software to stretch or compress the second signal pulse compared to the first signal pulse.

The computer system may also include computer-implemented software to calculate a local strain ε applied on tissue and adjust the value of α accordingly.

The computer system may also include computer-implemented software to code the emitted signals. The computer system may include software code generator or controlled hardware code generator.

Conventional equipment may be use to implement the elastography method according to the invention. The equipment may comprise a computer, a transmitter emitting signals according to the invention towards tissue and a receiver of echoed signals from the tissue, means for applying a strain on tissue, such as a hand-held compressor or a compressor activated by a motor controlled by the computer. The computer may be linked to a cross-correlator receiving digitalized signal of the emitted and echoed signals towards and from the tissue. A monitor may display the elastographic image. Such an equipment is described in U.S. Pat. No. 5,474,070.

1. A method for performing elastography, the method comprising the steps of:
a) applying a first coded voltage waveform u₁(t) to a transducer to emit a first ultrasonic signal p₁(t) towards tissue, with p₁(t)=u₁(t)*h(t) being the point spread function (PSF) generated by the waveform u₁, where t denotes the time and symbol * denotes a convolution product, h(t) being the impulse response of the transducer;
b) recording a first echoed signal s₁(t) in response to said first emitted signal;
c) applying a strain on the tissue;
d) applying a second coded voltage waveform u₂(t) to the transducer to emit a second ultrasonic signal p₂(t) towards the tissue under strain with p₂(t)=u₂(t)*h(t) being the point spread function (PSF) generated by the waveform u₂,
said second emitted signal being linked to said first emitted signal with the following relation:
p₂(t)=p₁(αt),
with a =1/(1 +), F being an initial estimate of the strain applied to the tissue;
e) recording a second echoed signal s₂(t) in response to said second emitted signal;
f) processing said second echoed signal according to the following relation:
s₂″(t)=s₂(t/α);
g) cross-correlating the first echoed signal s₁(t) and the processed second echoed signal s″₂(t) to provide an elastographic image of said tissue.

2. The method according to claim 1, wherein the strain applied to the tissue in step c) is negative, providing a compression of said tissue.

3. The method according to claim 1, wherein the strain applied to the tissue in step c) is positive, providing an expansion of said tissue.

4. The method according claim 1, wherein the first and second emitted signals are burst signals.

5. The method according claim 1, wherein the first emitted signal p₁(t) is pre-stretched so that its point spread function (PSF) duration is increased.

6. The method according claim 1, wherein the second signal p₂(t) is emitted with a predetermined value of α in step d), providing an elastographic image in step g);
the method further comprising the steps of:
h) calculating a local strain ε applied on tissue from the results of said elastographic image;
i) adjusting the value of α according to the calculated local strain ε;
j) repeating steps d) to g) with an adjusted value of α.

7. The method according to claim 6, wherein steps h) to j) are repeated iteratively until the adjusted value of a is substantially equal to the value applied to the second emitted signal p₂(t).

8. A system for performing the elastography method according to any claim 1, comprising:
hardware or computer-implemented software to link the second signal to be emitted to the first emitted signal;
hardware or computer-implemented software to cross-correlate the echoed signals.

9. The system according to claim 8, further comprising:
hardware or computer-implemented software to calculate a local strain ε applied on tissue and adjust the value of α accordingly.

10. The system according to claim 8, further comprising:
hardware or computer-implemented software to code the emitted signals.
